(19) United States
(12) Patent Application Publication
(10) Pub. No.: US 2024/0100276 A1
(43) Pub. Date: Mar. 28, 2024

(54) METHODS AND SYSTEMS OF SUPPLYING THERAPEUTIC GAS WITH IMPROVED MOUTH SENSING

(71) Applicant: INCOBA, LLC, Chesterfield, MO (US)

(72) Inventor: Alonzo C. AYLSWORTH, Wildwood, MO (US)

(73) Assignee: INCOBA, LLC, Chesterfield, MO (US)

(21) Appl. No.: 17/955,136

(22) Filed: Sep. 28, 2022

(51) Int. Cl. A61M 16/00; A61M 16/06

(52) U.S. Cl. CPC A61M 16/024; A61M 16/0666; A61M 2016/0027

(57) ABSTRACT

Supplying therapeutic gas with improved mouth sensing. At least one example is a system and related method of treating CPAP non-compliance by delivery of therapeutic gas during and after sleep apnea events. Another example is a system and related method to change the volume of therapeutic gas delivered responsive to changes in oral resistance to the airflow associated with the transitions from wakefulness to sleep. Yet still other examples are systems and related methods of selective delivery of the therapeutic gas in which the left and right sides of the mouth are measured and delivered to independently to take into account respiratory airflow may not be evenly distributed across the mouth.

### CROSS-REFERENCE TO RELATED APPLICATIONS

None.

### BACKGROUND

Patients with respiratory ailments may be required to breathe a therapeutic gas, such as oxygen. The therapeutic gas may be delivered to the patient from a therapeutic gas source by way of a nasal cannula. In some cases, the therapeutic gas is delivered to a patient continuously. That is, in continuous delivery the therapeutic gas is supplied at a constant flow rate throughout the patient's breathing cycle (i.e., both inhalation and exhalation). In other cases, such as to reduce consumption of the therapeutic gas, the therapeutic gas may be delivered in a bolus form at the beginning of each inhalation.

Any system or method that improves patient compliance and/or increases oxygen saturation of the patient may provide better patient outcomes.

### BRIEF DESCRIPTION OF THE DRAWINGS

For a detailed description of example embodiments, reference will now be made to the accompanying drawings in which:

FIG. 1 shows a view a patient wearing a related-art cannula;

FIG. 2 shows a view a patient wearing a related-art cannula;

FIG. 3 shows a system in accordance with at least some embodiments;

FIG. 4 shows a conserver in accordance with at least some embodiments;

FIG. 5 shows a conserver in accordance with at least some embodiments;

FIG. 6 shows, in block diagram form, the conserver of FIG. 4 in a shorthand notation;

FIG. 7 shows, in block diagram form, the conserver of FIG. 5 in a shorthand notation;

FIG. 8 shows, in block diagram form, an example conserver in which the narial sensors are pressure sensors and the oral sensors are flow sensors, in accordance with at least so embodiments;

FIG. 9 shows, in block diagram form, an example conserver in which the narial sensors are flow sensors and the oral sensors are pressure sensors, in accordance with at least so embodiments;

FIG. 10 shows, in block diagram form, an example conserver having both pressure and flow sensors associated with each hose connection, in accordance with at least so embodiments;

FIG. 11 shows a method in accordance with at least so embodiments; and

FIG. 12 shows a method in accordance with at least so embodiments

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . . ” Also, the term “couple” or “couples” is intended to mean either an indirect or direct connection. Thus, if a first device couples to a second device, that connection may be through a direct connection or through an indirect connection via other devices and connections.

“About,” in reference to a recited value, shall mean the recited value plus or minus+/−ten percent (10%) of the recited value.

“Assert” shall mean changing the state of a Boolean signal. Boolean signals may be asserted high or with a higher voltage, and Boolean signals may be asserted low or with a lower voltage, at the discretion of the circuit designer. Similarly, “de-assert” shall mean changing the state of the Boolean signal to a voltage level opposite the asserted state.

“Nares” shall mean the nostrils of a patient.

“Naris” shall mean a single nostril of a patient, and is the singular of “nares.”

“Sleep apnea event” shall mean, during sleep, an apnea event (e.g., pauses in breathing) and/or a hypopnea event (e.g., periods of shallow breathing).

“At least one of [X₁], [X₂], . . . and [X_{N}]” shall mean any of the X entries singly, any combination of the X entries being less than all the entries, or all the X entries.

“Controller” shall mean, alone or in combination, individual circuit components, an application specific integrated circuit (ASIC), a microcontroller with controlling software, a digital signal processor (DSP), a processor with controlling software, a programmable logic device (PLD), a field programmable gate array (FPGA), or a programmable system-on-a-chip (PSOC), configured to read inputs and drive outputs responsive to the inputs.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art understands that the following description has broad application, and the discussion of any embodiment is meant only to be exemplary of that embodiment, and not intended to intimate that the scope of the disclosure, including the claims, is limited to that embodiment.

Many patients are provided therapeutic gas to address respiratory ailments, such as chronic obstructive pulmonary disease (COPD), pulmonary fibrosis, oxygen desaturation issues associated with illness (e.g., SARS-CoV-19), or sleep disorders (e.g., central or obstructive sleep apnea). In situations where the supply of therapeutic gas is effectively limitless (e.g., hospitals), patients may be provided therapeutic gas continuously. In particular, the patient may wear a nasal cannula that has outlet ports or prongs that are associated, one each, with the nares of the patient. The therapeutic gas may flow continuously to the nares at a predetermined flow rate (e.g., 3 liters per minute (LPM)). Regardless of whether the patient is inhaling or exhaling, the therapeutic gas continuously flows, and thus delivery by this method is referred to as “continuous flow” or in a “continuous-flow mode.” A similar situation may exist for non-ambulatory patients in the home environment, where the therapeutic gas is provided from an oxygen concentrator. In the home environment with an oxygen concentrator, and regardless of whether the patient is inhaling or exhaling, the therapeutic gas may be provided in the continuous-flow mode.

In many cases, the therapeutic gas provided is oxygen. Oxygen is a corrosive gas, and exposing a patient to too much oxygen has detrimental effects, such as sores, scabbing, and swelling of the mucosal tissue of the nose and mouth. The sores, scabbing, and swelling may reduce the patency of the airways, thus further exacerbating oxygen desaturation issues. Thus, to reduce oxygen exposure some patients may be provided oxygen in a short burst or bolus at the beginning of each inhalation (e.g., during the first 100 milliseconds (ms) of the inhalation). Delivery by this method is referred to as “bolus flow” or in a “bolus-flow mode.” Providing therapeutic gas in as bolus, rather than continuously, may also be beneficial for ambulatory patients whose therapeutic gas is provided by a small portable gas cylinder. That is, ambulatory patients may use a small portable gas cylinder and a delivery device which senses and delivers a bolus of therapeutic gas with each inhalation. Because the delivery device reduces or conserves oxygen compared to continuous flow systems, such delivery devices may be referred to as conservers. Further, gas cylinders, even when low, may have several hundred pounds of pressure. The bolus of therapeutic gas delivered by a conserver thus has relatively high pressure (e.g., 20 pounds per square inch (PSIG) or more) which provides a high velocity burst to help force the bolus into the lungs of the patient.

The volume of therapeutic gas delivered is selected by a clinician during patient titration. In many cases, the patient is titrated with a continuous flow of therapeutic gas, and when the patient uses a conserver the volume of therapeutic gas provided by each bolus is controlled or set using a known relationship between continuous flow prescription flow rate and bolus-flow mode volume. In particular, in situations where the patient is titrated using continuous flow, during the bolus flow the patient is provided, at each inhalation, about 16.5 milliliters (mL) of volume for every 1 LPM of continuous flow prescription flow rate. In other cases, the patient may be titrated using bolus-flow, and thus the prescription flow rate may come directly from the patient titration.

Various examples are directed to methods and systems of providing therapeutic gas to a patient with improved mouth sensing. One example is a nasal-oral cannula that comprises separate and fluidly isolated sensing/delivery paths for the left naris, the right naris, the left portion of the mouth, and the right portion of the mouth. The example nasal-oral cannula may be associated with a therapeutic gas delivery device that can separately sense airflow through each of the left naris, the right naris, the left portion of the mouth, and the right portion of the mouth, and the example therapeutic gas delivery device is designed and constructed to deliver therapeutic gas to any one or a combination of the left naris, the right naris, the left portion of the mouth, and the right portion of the mouth. An example therapeutic gas delivery system (e.g., the nasal-oral cannula and the delivery device) enables several advances in therapeutic gas therapies. For example, the therapeutic gas delivery system may be used in cases of patient non-compliance with continuous positive airway pressure (CPAP) systems—such as sensing sleep apnea events and providing therapeutic gas responsive to the sleep apnea event and/or for a predetermined time thereafter. Providing oxygen during the sleep apnea event and/or for a predetermined time thereafter reduces the chances of lowered oxygen saturation, even if that lowered oxygen saturation does not fall to the level of clinical oxygen desaturation As another example, the therapeutic gas delivery system enables detection of a patient transitioning to sleep, and taking action to reduce the chances of lowered oxygen saturation associated with the sleep, even if that lowered oxygen saturation does not fall to the level of clinical oxygen desaturation. The specification now turns to a discussion of why related-art cannulas fall short.

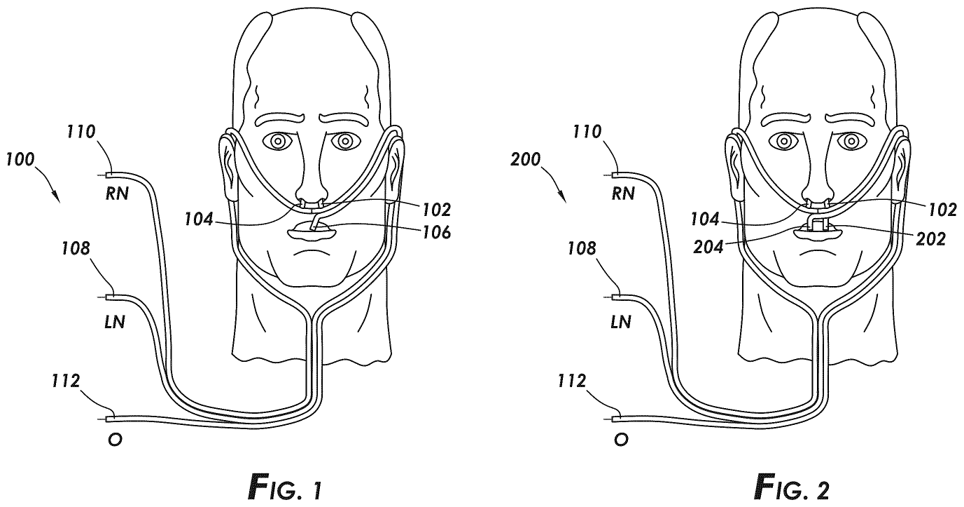

FIG. 1 shows a view a patient wearing a related-art cannula. In particular, the example cannula 100 comprises a left narial prong 102 associated with the left naris, a right narial prong 104 associated with the right naris, and a mouth prong 106 associated with and approximately centered along the patient's mouth. The left narial prong 102 is fluidly coupled to a left narial tube 108, the right narial prong 104 is fluidly coupled to a right narial tube 110, and the mouth prong 106 is fluidly coupled to an oral tube 112. In theory, each prong resides within the airflow through its respective breathing orifice, and enables an attached device to detect airflow associated with respiration through the respective breathing orifice. However, use of a single oral prong has shortcomings in the detection of oral airflow. For example, when a patient is sleeping on their side, the lower side of the mouth may be closed, while the upper side of the mouth may be open. Thus, when the respiratory airflow flows partially or fully through the mouth, use of the centered mouth prong may miss some or all the airflow through the mouth.

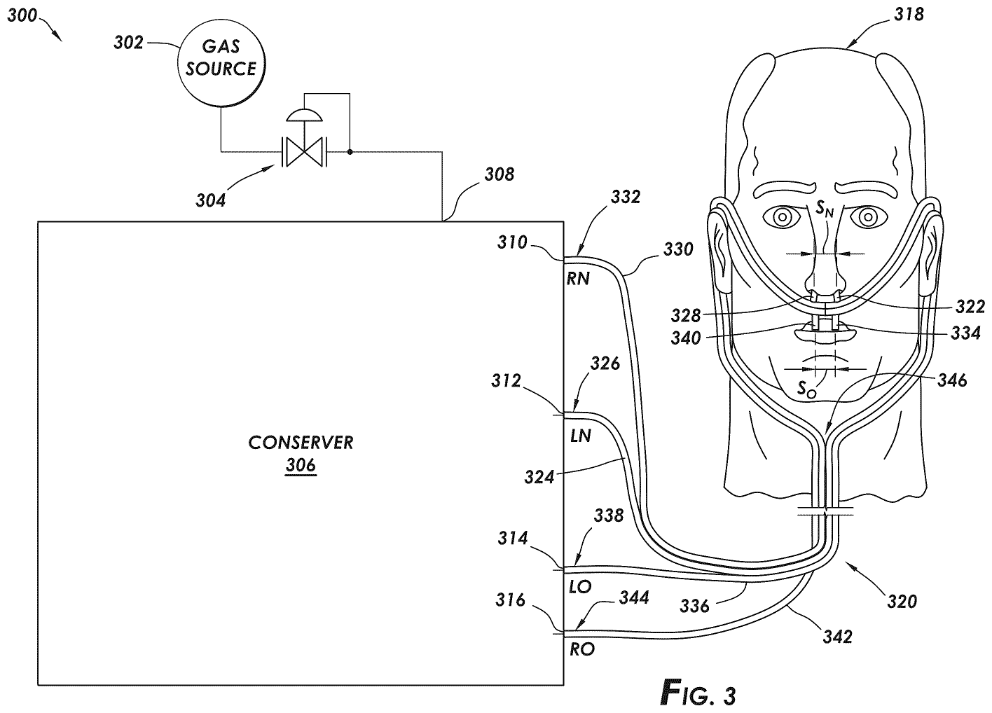

Related-art cannulas and systems attempt to address the issue by use of multiple mouth prongs; however, considering the mouth is a single breathing orifice, the related-art cannulas and systems treat the mouth as a single sensed entity. FIG. 2 shows a view a patient wearing a related-art cannula having multiple mouth prongs associated with a single oral tube. In particular, the example cannula 200 comprises the left narial prong 102 associated with the left naris, the right narial prong 104 associated with the right naris, a left mouth prong 202 associated with the left side of the mouth, and a right mouth prong 204 associated with the right side of the mouth. The left narial prong 102 is fluidly coupled to the left narial tube 108 and the right narial prong 104 is fluidly coupled to the right narial tube 110. Both the left mouth prong 202 and the right mouth prong 204 are fluidly coupled to the oral tube 112. That is say, the single oral tube 112 is fluidly coupled to both the left mouth prong 202 and the right mouth prong 204. It turns out, however, that the arrangement of FIG. 2 makes sensing airflow through the mouth less sensitive rather than more sensitive.

Consider first a situation in which the respiratory airflow of the patient flows partially or fully through the patient's mouth, and that the airflow through the mouth is evenly divided across the mouth from left to right. When the patient inhales, the reduced pressure associated with the inhalation draws air through the left mouth prong 202 and the right mouth prong 204 evenly. In particular, the portion of the inhalation airflow associated with the mouth is evenly divided between the left mouth prong 202 and the right mouth prong 204 (e.g., 50% left mouth prong, 50% right mouth prong). However, it is rare that airflow associated with the mouth is evenly distributed across the mouth. Again as an example, when the patient is sleeping on their side, the lower side of the mouth may be closed, while the upper side of the mouth may be open.

The inventor of the current specification found that imbalance of the airflow across the mouth adversely affects the ability of related-art cannulas to sample representative air flow, and thus adversely affects the ability of therapeutic gas delivery devices to sense and operate correctly. For purposes of highlighting the shortcomings of the arrangement of FIG. 2, now consider a situation in which the patient is sleeping on their side such that the right side of the mouth is closed, and the portion of the respiratory airflow through the patient's mouth flows exclusively through the left side of the mouth. During an example inhalation, a portion of the inhaled gas flows through the left mouth prong 202. However, because the right side of the mouth is fully closed in this example, the right mouth prong 204 is effectively at atmospheric pressure. The inhaled gas drawn through the left mouth prong 202 has two possible paths: 1) through the therapeutic gas delivery device (not shown), then through the length of the oral tube 112, and then through the left mouth prong 202 into the patient's mouth; or 2) through the right mouth prong 204, then through single lumen to the left mouth prong 202, and then through the left mouth prong 202 into the patient's mouth. The path from the right mouth prong 204 to left mouth prong 202 is a significantly shorter path and thus has lower resistance to airflow. In this example, a portion, in fact a substantial portion, of the inhaled gas that flows through the left mouth prong 202 “leaks” in through the right mouth prong 204 rather than flowing through the therapeutic gas delivery device and the oral tube 112. Of course, when the right side of the patient's mouth is open and the left side of the patient's mouth is closed, a similar “leak” occurs from the left mouth prong 202 to the right mouth prong 204. The “leak” may also occur in situations in which one side of the mouth is only slightly open to flow. Thus, for an attached therapeutic gas delivery device that senses oral airflow or pressure associated with oral airflow, the addition of the second mouth prong makes the overall system less sensitive rather than more sensitive. The specification now turns to an example system.

FIG. 3 shows an example system 300. The example system 300 comprises a gas source 302. The gas source 302 may take any suitable form, such as a therapeutic gas cylinder. Because the pressure within the example therapeutic gas cylinder may be from several hundred pounds per square in gauge (PSIG) to several thousand PSIG, the gas source 302 may be associated with a pressure regulator 304. The pressure regulator 304 reduces the pressure of the therapeutic gas provided to the delivery system (discussed more below) to below 30 PSIG, in some cases to 25 PSIG and below, and in example cases between and including 20 PSI and 25 PSIG.

The therapeutic gas downstream of the pressure regulator 304 is fluidly coupled to a therapeutic gas delivery device or conserver 306. The example conserver 306 defines a source-hose connection 308 fluidly coupled to the pressure regulator 304 and the gas source 302. The example conserver 306 also defines a first-hose or right-naris hose connection 310 and a second-hose or left-naris hose connection 312. The example conserver 306 also defines a third-hose or left-oral hose connection 314 and a fourth-hose or right-oral hose connection 316.

In the example system, the conserver 306 is fluidly coupled to a patient 318 by way of a nasal-oral cannula 320 having a dual-lumen or bifurcated nasal portion and a dual-lumen or bifurcated oral portion. In particular, the example nasal-oral cannula 320 defines a hose or tube associated with the left side of the mouth, and a separate and distinct hose or tube associated with the right side of the mouth. The example nasal-oral cannula 320 may also define a hose or tube associated with the right naris, and a separate and distinct hose or tube associated with the left naris. More particularly, the example nasal-oral cannula 320 defines a left narial prong 322 fluidly coupled to a distal end of a left narial tube 324, and a left narial-hose connector 326 disposed at a proximal end of the left narial tube 324. In some cases the left narial-hose connector 326 may be just the proximal end of the left narial tube 324 (e.g., that telescopes over a barbed connection defined by the conserver 306), but in other cases the left narial-hose connector 326 may be an industry standard hose connector, such as a luer connector or luer fitting. The example nasal-oral cannula 320 defines a right narial prong 328 fluidly coupled to a distal end of a right narial tube 330, and a right narial-hose connector 332 disposed at the proximal end of the right narial tube 330. As with the left narial-hose connector 326, the right narial-hose connector 332 may be just the proximal end of the right narial tube 330 or an industry standard hose connector. The right narial tube 330 is fluidly isolated from the left narial tube 324 even though the tubes may physically meet or intersect between the prongs. That is, the left narial tube 324 and the right narial tube 330 may meet, and their respective central axes intersect, between the left narial prong 322 and the right narial prong 328, but nevertheless are fluidly isolated. The left and right narial prongs 322 and 328 define a first spacing SN. The left and right narial prongs 322 and 328 may be parallel as manufactured, but given the flexible tubing nature of the tubes and the prongs, in use the left and right narial prongs 322 and 328 may be flexed toward each other as shown in FIG. 3 or away from each other depending upon the shape and size of the patient's nares.

Still referring to FIG. 3, the example nasal-oral cannula 320 further includes a left oral prong 334 fluidly coupled to a distal end of a left oral tube 336, and a left oral-hose connector 338 disposed at a proximal end of the left oral tube 336. In some cases the left oral-hose connector 338 may be just the proximal end of the left oral tube 336 or an industry standard hose connector. The example nasal-oral cannula 320 defines a right oral prong 340 fluidly coupled to a distal end of a right oral tube 342, and a right oral-hose connector 344 disposed at the proximal end of the right oral tube 342. As with the left oral-hose connector 338, the right oral-hose connector 344 may be just the proximal end of the right oral tube 342 or an industry standard hose connector. The right oral tube 342 is fluidly isolated from the left oral tube 336 even though the tubes may physically meet or intersect between the prongs. That is, the left oral tube 336 and the right oral tube 342 may meet, and their respective central axes intersect, between the left oral prong 334 and the right oral prong 340, but nevertheless are fluidly isolated. The left and right oral prongs 334 and 340 define a first spacing S_{O}, and in example cases the spacing S_{O} is the same or greater than the spacing S_{N}. The left and right oral prongs 334 and 340 may be parallel as manufactured, but given the flexible tubing nature of the tubes and the prongs, in use prongs may be flexed toward or away from each other.

In example cases, the left narial tube 324 is coupled to and runs parallel to the left oral tube 336 from a location of the left oral prong 334 to a merge point 346. The right narial tube 330 is coupled to and runs parallel to the right oral tube 342 from a location of the right oral prong 340 to the merge point 346. The left and right narial tubes 324 and 330, and the left and right oral tubes 336 and 342, couple to and run parallel to each other, in some cases parallel to each other greater than half a distance from the merge point 346 to the respective hose connectors 326, 332, 338, and 344. It follows that the tubes create a loop defined in first part by the combined left narial tube 324 and left oral tube 336, and the loop defined in second part by the combined right narial tube 330 and the right oral tube 342. In the example use case of FIG. 3, the loop enables the nasal-oral cannula 320 to be placed in operational relationship to the patient's 318 nose and mouth, run over the patient's ears, and then have the merge point 346 reside beneath the patient's chin. However, in other cases, the merge point 346 may be disposed behind the patient's head, and the size of the loop (e.g., circumference) may be designed or adjusted accordingly.

In the example system, the left-narial hose connector 326 is coupled to the left-naris hose connection 312 defined by the conserver 306 such that the conserver 306 can sense respiratory airflow through the left naris and couple the source-hose connection 308 to the left naris connection 312 to deliver therapeutic gas from the gas source 302. The right-narial hose connector 332 is coupled to the right-naris hose connection 310 defined by the conserver 306 such that the conserver 306 can sense respiratory airflow through the right naris and couple the source-hose connection 308 to the right naris connection 310 to deliver therapeutic gas from the gas source 302. The left oral-hose connector 338 is coupled to the left-oral hose connection 314 defined by the conserver 306 such that the conserver 306 can sense respiratory airflow through the left side of the mouth and couple the source-hose connection 308 to the left-oral hose connection 314 to deliver therapeutic gas from the gas source 302. And finally, the right-oral hose connector 344 is coupled to the right-oral hose connection 316 defined by the conserver 306 such that the conserver 306 can sense respiratory airflow through the right side of the mouth and couple the source-hose connection 308 to the right oral connection 316 to deliver therapeutic gas from the gas source 302.

The sensing of respiratory airflow and providing therapeutic gas by the conserver 306 likewise may take many forms. In one example, the conserver 306 senses inhalations at one or a combination of the various sensing locations (e.g., left naris, right naris, left mouth, and right mouth), and delivers therapeutic gas as a bolus at the beginning of each inhalation (e.g., bolus-flow mode). In some cases, the bolus may be provided evenly to all sensing locations at which the inhalation is sensed. In other cases, the bolus may be delivered in proportion to the rate of inhalation airflow sensed at each sensing location. In yet still other cases in which inhalations are sensed at two more sensing locations, the conserver 306 may deliver therapeutic gas at only one location, and change the delivery location periodically (e.g., every breath, every third breath) to reduce the drying and irritation of mucosal tissue at any one delivery location.

Still considering delivery of therapeutic gas by the conserver 306, in another examples the conserver 306 may deliver therapeutic gas in a continuous-flow mode to one or more of the sensing locations. For example, the conserver 306 may deliver therapeutic gas in the continuous-flow mode to all the sensing locations for a period of time (e.g., three minutes), and then cease delivery and sense inhalations at the sensing location(s) before continuing again to deliver in the continuous-flow mode. In other cases, the conserver 306 may deliver therapeutic gas to the most open sensing location in a continuous-flow mode while sensing respirations at other sensing location(s). If the conserver 306 determines the most open airway changed, the conserver 306 may then shift to delivery of therapeutic gas in the continuous-flow mode to most open airway while sensing respirations at the other sensing locations.

In yet another example, the conserver 306 may deliver therapeutic gas using a combination of bolus-flow mode at some sensing locations, and continuous-flow mode at other sensing locations. For example, if at least one naris is open to flow, and at least one side of the mouth is open to flow, then the example conserver 306 may provide therapeutic gas in the continuous-flow mode to the side of mouth open to flow while providing therapeutic gas to the open naris in the bolus-flow mode at the beginning of each inhalation. The example of bolus-flow mode to a naris and continuous-flow mode to mouth may be reversed in some situations—continuous-flow mode to one or both nares, and bolus-flow mode to sensing location(s) associated with the mouth.

Yet still further example conservers 306 may deliver therapeutic gas in a modified continuous-flow mode—sometimes referred to as pulse-flow continuous mode. In particular, in this example the conserver 306 may deliver therapeutic gas to the patient during inhalation, with the delivery in a continuous-flow mode during each respiration, but where the continuous flow has a predetermined duration that ends during each respiration such that the predetermined duration is based on a ratio of a duration of a previous inhalation to the duration a previous exhalation. The pulse-flow continuous mode can be used with respect to any one, all, or a combination of the sensing locations. The pulse-flow continuous mode may be distinguished from the bolus-flow mode in that the rate of therapeutic gas flow during the pulse-mode continuous flow is based on a continuous-flow mode prescription flow rate, and the delivery volume of each delivery exceeds the relationship of about 16.5 ml of bolus volume for every 1 LPM of continuous-flow mode prescription flow rate. Further still, the pulse-flow continuous mode may be used in combination with the bolus-flow mode—such as pulse-flow continuous mode to the mouth and bolus-flow mode to at least one naris.

As noted above, the inventor of the current specification found that an imbalance of the airflow across the mouth adversely affects the ability of related-art cannulas to sample representative airflow, and thus adversely affects the ability of related-art devices to sense and operate correctly. By using separate and distinct oral tubes 336 and 342 associated with the oral prongs 334 and 340, respectively, the shortcomings of the “leak” from prong-to-prong is reduced or eliminated. The reduction or elimination of the “leak” increases the sensitivity of the airflow detection through the mouth, in some cases the increase in sensitivity is between and including 4 and 16 times compared to the single-plenum oral sensing of FIG. 2. The significantly increased sensitivity enables new therapies, discussed in greater detail below.

The first new therapy enabled by the increased sensitivity is a therapy that may be used in the case of CPAP non-compliance. That is, CPAP patients cease using their CPAP devices for a variety of reasons, such as the mask being uncomfortable, leaks of air waking the patient, or the noise of the CPAP device keeping the patient or the patient's spouse awake. One potential therapy enabled by the increased sensitivity may be applicable in the case of CPAP non-compliance. That is, rather than sleep with a CPAP machine and related CPAP mask, the patient sleeps with a system as shown in FIG. 3—a gas source 302 coupled to a conserver 306, and the conserver 306 coupled to the patient 318 by way of the nasal-oral cannula 320. In the example therapy, the conserver 306 initially does not provide therapeutic gas to the patient 318. Rather, the conserver 306 senses the respiratory airflow at the various sensing locations as the patient sleeps. In the absence of a sleep apnea event (e.g., regular tidal breathing of the patient), the conserver 306 refrains from providing therapeutic gas to the patient and continues to monitor the patient. Stated otherwise, the conserver 306 senses pre-apnea inhalations, and refrains from providing or does not provide therapeutic gas to the patient during the pre-apnea inhalations. When the conserver 306 senses a sleep apnea event (e.g., a hypopnea or apnea), the conserver 306 is designed and constructed to provide therapeutic gas to the patient 318 at least during inhalations that occur after the sleep apnea event—such as at the breakthrough breath and for a plurality of inhalations after the breakthrough breath. Stated otherwise, the conserver 306 senses a sleep apnea event, then senses post-apnea inhalations, and delivers therapeutic gas to the patient for a plurality of the post-apnea inhalations.

The response of the conserver 306 to the detection of the sleep apnea event may take many forms. The discussion is divided into periods of time during the sleep apnea event, and periods of time after the sleep apnea event (i.e., during the post-apnea inhalations). Turning first to periods of time during the sleep apnea event. In example cases the conserver 306 is designed and constructed to refrain from delivering therapeutic gas to the patient 318 during the sleep apnea event. The theory of operation associated with refraining from delivery during the sleep apnea event is that if the patient is not breathing, or only breathing shallowly, any therapeutic gas provided will not have a therapeutic effect because the gas does not travel into, or far enough into, the lungs of the patient.

Still considering periods of time during the sleep apnea event, in yet still other cases the conserver 306 is designed and constructed to deliver therapeutic gas to the patient 318 during the sleep apnea event. That is, after determining the presence of the sleep apnea event and before the post-apnea inhalations (e.g., before the breakthrough breath), the conserver 306 is designed and constructed to deliver therapeutic gas to at least one of the various sensing locations. The theory of operation associated with delivering therapeutic gas during the sleep apnea event is that the patient's nose and/or mouth is flooded with therapeutic gas such that, when the breakthrough breath occurs, the breakthrough breath is predominately of therapeutic gas.

The selection of location at which to provide the therapeutic gas during the sleep apnea event may take many forms. In one example, the conserver 306 may be designed and constructed to provide therapeutic gas to all the sensing locations (e.g., left naris, right naris, left mouth, and right mouth). In other cases, the conserver 306 may be designed and constructed to provide the therapeutic gas to the sensing location(s) that most recently carried the respiratory airflow. For example, if in the last pre-apnea inhalation the patient inhaled predominantly through the left naris and the left mouth, then the therapeutic gas provided during the sleep apnea event may be provided exclusively to the left naris and left mouth.

Notwithstanding the variations in location of delivery during the sleep apnea event, the conserver 306 may be designed and constructed to deliver therapeutic gas for various amounts of time during the sleep apnea event. For example, the conserver 306 may deliver therapeutic gas during the entire sleep apnea event. The conserver 306 may deliver therapeutic gas for a duration sufficient to provide a predetermined volume of therapeutic gas. The conserver 306 may deliver therapeutic gas for a duration being greater than half a duration of a previous sleep apnea event. The conserver 306 may deliver for a duration equal to the duration of a previous sleep apnea event. The conserver 306 may deliver therapeutic gas during the sleep apnea event for a predetermined duration (e.g., three seconds). Regardless of the precise criteria for delivery, the patient is provided therapeutic gas such that, at the breakthrough breath, therapeutic gas is present.

Still considering delivery of therapeutic gas during the sleep apnea event, the volume and delivery time may take many forms. In one example, the conserver 306 may be designed and constructed to deliver therapeutic gas in a continuous-flow mode once the sleep apnea event is detected. That is, the conserver 306 may sense the final pre-apnea inhalation and/or the final pre-apnea exhalation, and based on the absence of a follow-on inhalation within a predetermined period of time (e.g., 10 second or more, in some cases with an actual or suspected oxygen desaturation of the patient), the conserver 306 may be designed and constructed to deliver therapeutic gas in the continuous-flow mode to some or all the sensing locations. In the case of delivery in the continuous-flow mode to all the sensing locations, the conserver 306 may be further designed and constructed to periodically to cease delivery and determine whether the patient resumed breathing thus ending the sleep apnea event.

Still considering delivery of therapeutic gas during the sleep apnea event, the conserver 306 may be designed and constructed to provide a plurality of pulses of therapeutic gas to the patient (e.g., pulses of 100 ms duration). That is, during the sleep apnea event the conserver 306 quickly turns on and off the flow of therapeutic gas to the patient, such as two pulses per second or greater, and in one case between and including three to six pulses per second. The conserver 306 may be designed and constructed to provide the pulses of therapeutic gas to one sensing location, less than all the sensing locations, or all the sensing locations. The theory of operation of pulsing the therapeutic gas during the sleep apnea event may be twofold. First, and as before, providing the therapeutic gas floods the breathing orifices with therapeutic gas such that, at the breakthrough breath, therapeutic gas is present and inhaled. Second, the pulses of therapeutic gas may partially wake the patient (e.g., cause a brain arousal) sufficient to trigger the breakthrough breath, thus ending the sleep apnea event early, perhaps before the patient experiences oxygen desaturation.

Still considering use of the system of FIG. 3 in the case of CPAP non-compliance, and turning now to periods of time after the sleep apnea event (i.e., during the post-apnea inhalations). In example cases, once the conserver 306 senses the end of the sleep apnea event (e.g., senses post-apnea inhalations), the conserver 306 may be designed and constructed to deliver therapeutic gas to the patient for a plurality of post-apnea inhalations. That is, upon sensing post-apnea inhalations, the conserver 306 may deliver in a bolus-flow mode for a predetermined period of time (e.g., 10 minutes, or a predetermined number of inhalations) to ensure the patient's blood oxygen saturation recovers from the sleep apnea event, and then cease delivery and begin monitoring the patient for a subsequent sleep apnea event. In other cases, upon sensing post-apnea inhalations the conserver 306 may deliver in a continuous-flow mode for a predetermined period of time (e.g., 10 minutes, or a predetermined number of inhalations) to ensure the patient's blood oxygen saturation recovers from the sleep apnea event, and then cease delivery and begin monitoring the patient for a subsequent sleep apnea event. That is, after delivery of therapeutic gas for a plurality of post-apnea inhalations, the conserver 306 may be designed and constructed to cease delivering therapeutic gas, and refrain from delivering therapeutic gas during further inhalations unless and until a subsequent sleep apnea event occurs. More generally, the conserver 306 may deliver in any delivery mode discussed above (e.g., bolus-flow mode, continuous-flow mode, pulse-flow continuous mode), and in any combination, to the various breathing orifices open to respiratory airflow. Thereafter, the conserver 306 may be designed and constructed to cease delivering therapeutic gas, and refrain from delivering therapeutic gas during further inhalations unless and until a subsequent sleep apnea event occurs. When a subsequent sleep apnea event occurs, the conserver 306 again operates as described in any of the variations above.

As noted above, the inventor of the current specification found that an imbalance of the airflow across the mouth adversely affects the ability of related-art cannulas to sample representative airflow. By using separate and distinct oral tubes 336 and 342 associated with the oral prongs 334 and 340, respectively, the shortcomings of the “leak” from prong-to-prong created by the imbalanced airflow is reduced or eliminated. The reduction or elimination of the “leak” may increase the sensitivity of the airflow detection associated with mouth. The significantly increased sensitivity enables new therapies, such as addressing changes in resistance to airflow of the nasopharynx of the patient during the transition from being awake or wakefulness to sleep. Before delving into specifics of the further example therapy, the specification terms to issues of nasal cycle and oral resistance to airflow.

Humans experience a phenomenon termed “nasal cycle” in which the naris most open to airflow changes over the course of minutes to hours. For example, temporary swelling the turbinates associated with one naris may cause an increase in resistance to airflow through that naris, reducing or blocking airflow. In some cases, the naris most open to airflow shifts back and forth over time. At certain points in the shifting back and forth, both nares may be open to flow, and at other points in the shifting both nares may be substantially blocked. That is, if the turbinates associated with both nares are swollen simultaneously, the patient may be unable to breathe through nose, and during such times the patient may breathe through the mouth. Hold that thought.

With respect to oral resistance to airflow, the inventor of the present specification believes that as a patient transitions from wakefulness to sleep, certain muscles of the body relax—including certain muscles in the neck. The relaxation of the muscles in the neck may cause the cross-sectional flow area in the throat to decrease. The relaxation of the muscles may be part of a larger physical relaxation of the patient in the transition from wakefulness to sleep, or may be attributable to sleep paralysis associated with rapid eye movement (REM) sleep. Regardless of the precise physiological reason, the result is an increase resistance to airflow through the mouth.

Now consider nasal cycle together with issues of increased oral resistance to airflow in the transition from wakefulness to sleep. In particular, consider a patient breathing fully or partially through the mouth. As a patient transitions from wakefulness to sleep, the oral resistance to airflow increases. The increase in oral resistance to airflow results in less airflow into the lungs for the same inspiratory effort. If the transition from wakefulness to sleep occurs during a period of time in which neither naris is open to flow as caused by nasal cycle, the reduction in airflow to the lungs is even more pronounced. For patients receiving therapeutic gas through the mouth to maintain oxygen saturation, the increased oral resistance to airflow results in less therapeutic gas to the patient and thus an increased risk of oxygen desaturation, even if that desaturation does not fall to the level of clinical desaturation.

Thus, another example therapy enabled by the increased sensitivity associated with the mouth is a system in which the volume or amount of therapeutic gas provided to the patient is increased based on discerning that the patient transitioned from wakefulness to sleep. Consider again a situation such as shown FIG. 3—a gas source 302 coupled to a conserver 306, and the conserver 306 coupled to the patient 318 by way of the nasal-oral cannula 320. Further consider that the patient 318 receives therapeutic gas to some or all the sensing locations with each breath (e.g., bolus-flow mode, continuous-flow mode, pulse-flow continuous mode). In accordance with various examples, the conserver 306 is designed and constructed to sense pre-sleep inhalations through the mouth of the patient (e.g., by way of the left oral prong 334 and/or the right oral prong 340), and to deliver a first volume of therapeutic gas to the mouth of the patient during each inhalation. In some cases, the patient 318 may be breathing exclusively through the mouth, but in other cases the patient 318 may be breathing predominantly through the mouth. The sensing and delivery may continue for many pre-sleep inhalations.

To implement the example therapy, the conserver 306 is further designed and constructed to discern a transition of the patient 318 from wakefulness to sleep (i.e., a transition to a sleep state), with inhalations during sleep referred to as sleep inhalations. Discerning the transition from wakefulness to sleep may take many forms. For example, the transition from wakefulness to sleep may manifest itself as a change in respiration of the patient (e.g., decreased respiration rate, increased respiration duration, decreased respiration effort, increase in exhalation duration). In other cases, the transition from wakefulness to sleep may manifest itself in an analysis of a plurality of respirations sensed by the conserver 306. For example, a plurality of respiration waveforms may be cross-correlated, with a period of low cross-correlation followed by a period of high cross-correlation indicating a transition from wakefulness to sleep. As another example, a plurality of respiration waveforms may be subjected to Fourier Transform analysis, and with a shift in the fundamental frequency over time indicating a transition from wakefulness to sleep, such as described in U.S. Pat. No. 5,845,636. Thus, in sensing respirations of the patient 318, the conserver 306 may discern the transition from wakefulness to sleep. It is noted, however, that discerning the transition from wakefulness to sleep may occur over several respirations. The patient 318 may physiologically transition to sleep at a certain point in time, but because the conserver 306 may analyze a plurality of respiration waveforms as part of the analysis, discerning that the patient 318 transitioned from wakefulness to sleep may take place after the physiological transition from wakefulness to sleep. Nevertheless, in example cases the conserver 306 may discern the transition from wakefulness to sleep within 40 respiration the physiological transition, in some cases within 20 respirations of the physiological transition, and in a particular case within 10 respirations of the physiological transition. It follows that discerning the transition from wakefulness to sleep shall not be read to require determining the precise moment in time when physiological transition occurs.

Regardless of how the conserver 306 discerns the transition from wakefulness to sleep, the conserver 306 is designed and constructed to deliver a second volume of therapeutic gas to the mouth of the patient during each sleep inhalation, with the second volume being larger than the first volume. The second volume being larger than the first volume may take many forms depending on the delivery mode of the conserver 306 during the pre-sleep inhalations. For example, if the conserver 306 was delivering therapeutic gas in the bolus-flow mode at each pre-sleep inhalation, and each bolus of therapeutic gas had a first volume, after discerning the transition from wakefulness to sleep the conserver 306 may be designed and constructed to deliver therapeutic gas during each sleep inhalation in the bolus-flow mode but with each bolus having a second volume larger than the first volume.

As another example of differences in volume, if the conserver 306 was delivering therapeutic gas in the continuous-flow mode during each pre-sleep inhalation, a first flow rate of the continuous flow over the duration of each pre-sleep inhalation resulted in a first volume during each pre-sleep inhalation. After discerning the transition from wakefulness to sleep, the conserver 306 may be designed and constructed to deliver therapeutic gas in the continuous-flow mode, but with an increased second rate flow compared to the first flow rate. Thus, the second flow rate of the continuous flow over the duration of each sleep inhalation results in a second volume greater than the first volume delivered during each sleep inhalation.

As another example of differences in volume, if the conserver 306 was delivering therapeutic gas in the pulse-flow continuous mode during each pre-sleep inhalation, a first flow rate of the flow over the delivery time resulted in a first volume during each pre-sleep inhalation. After discerning the transition from wakefulness to sleep, the conserver 306 may be designed and constructed to deliver therapeutic gas in the pulse-flow continuous mode but with an increased flow rate and/or an increased delivery time. Thus, the increased flow rate and/or the increased delivery time results in a second volume greater than the first volume delivered during each sleep inhalation.

In yet still further cases, delivery of the second volume during the sleep inhalations may involve a change a delivery mode. For example, if the conserver 306 was delivering therapeutic gas in bolus-flow mode at each pre-sleep inhalation, and each bolus of therapeutic gas had a first volume, after discerning the transition from wakefulness to sleep the conserver 306 may be designed and constructed to deliver therapeutic gas during each sleep inhalation in the continuous-flow mode or pulse-flow continuous mode (as described above), and thus volume the volume of therapeutic gas delivered during each sleep inhalation is greater than the volume of each bolus delivered during the pre-sleep inhalations.

Still referring to FIG. 3, use of the example nasal-oral cannula 320 potentially results in sensing airflow, and delivering therapeutic gas, at multiple locations with respect to the mouth of the patient 318. For example, sensing the sleep inhalations through the mouth by way of the nasal-oral cannula 320 may comprise the left oral prong 334 sensing airflow between the left oral commissure and the left philtral ridge. In addition to or in place of the sensing location associated with the left oral prong 334, sensing the sleep inhalations through the mouth by way of the nasal-oral cannula 320 may comprise the right oral prong 340 sensing airflow between the right oral commissure and the right philtral ridge. It follows from the sensing locations that the delivery of therapeutic gas to the mouth may be similarly located. In particular, if airflow is sensed between the left oral commissure and the left philtral ridge by the left narial prong 334, delivery of therapeutic gas through the left narial prong 334 will be between the left oral commissure and the left philtral ridge. If airflow is sensed between the right oral commissure and the right philtral ridge by the right narial prong 340, delivery of therapeutic gas through the right narial prong 340 will be between the right oral commissure and the right philtral ridge.

The example conserver 306 may be designed and constructed to be used both during wakefulness (e.g., ambulatory use during the day), and during sleep (e.g., daily naps, or overnight use). In some cases, use of the conserver 306 during the day may be by way of a nasal-only cannula (e.g., a bifurcated nasal cannula without mouth sensing). Use of the conserver 306 at night may be by way of a nasal-oral cannula, such as nasal-oral cannula 320. That is to say, as a patient readies for bed, the patient may disconnect the nasal-only cannula and connect a nasal-oral cannula (e.g., nasal oral cannula 320) to the conserver 306. It follows that the operational mode of the conserver 306 may be based on the type of cannula coupled to the conserver. For example, if a nasal-only cannula is coupled to the conserver 306, the conserver 306 may be designed and constructed to deliver only to the nasal hose connections 310 and 312. Relatedly, if a nasal-only cannula is coupled to the conserver 306, the conserver 306 may refrain from executing methods that attempt to discern transitions from wakefulness to sleep, and such refraining may thus reduce controller loading and therefore reduce battery usage.

In some cases, the cannula may be associated with a connector that both fluidly couples the tubes to the conserver 306, and conveys to the conserver 306 data indicative of the type of cannula attached. For example, U.S. Pub. 2007/0277824 titled “Hose Connection System for Narially Sensitive Diagnostic Devices” describes example systems. However, in other cases the example conserver 306 may be designed and constructed to determine whether a tube of a nasal cannula is connected to any particular hose connection (e.g., hose connections 310, 312, 314, and/or 316). Before describing examples of the conserver 306 determining whether a tube is connected, consider the following about tubes of cannulas. Tubes that define cannulas are made of polymeric material that is flexible and thus expands slightly when the pressure within the tube is above atmospheric pressure. Moreover, the tubes that define cannulas have a length, ranging from about two meters to between seven and 10 meters. It follows that each tube presents a resistance to gas movement through the tube (e.g., airflow, therapeutic gas).

With respect to the conserver 306 determining whether a tube is connected, and as a representative example, consider the left-oral hose connection 314 and the left oral tube 336. The conserver 306, at any appropriate time (e.g., initial power up, or responsive to an internal or external command), may test to determine whether the tube 336 is connected. In particular, the conserver 306 may test by providing a burst of therapeutic gas to the left-oral hose connection 314, measuring or reading data indicative of the gas flow of the therapeutic gas, and making a determination based on the data indicative of gas flow. The data indicative of gas flow may be the pressure of the therapeutic gas provided to the left-oral hose connection 314 over time. In one example, the presence or absence of a tube may be determined based on the rise time of the pressure measured and/or the peak pressure achieved. In the absence of a tube coupled to the representative left-oral hose connection 314, during the burst the measured pressure rises at a first rate and achieves a first peak pressure. However, when a tube is coupled to the representative left-oral hose connection 314, during the burst the measured pressure rises at a second rate, slower than the first rate, and achieves a second peak pressure higher than the first peak pressure. The differences in rise time for measured pressure may be attributable to the resistance to gas flow presented by the tube in combination with the “capacitive” or storage effects caused by expansion of the tube based on increased differential pressure across the tube. The differences in peak pressure may be attributable to the physical distance to the atmospheric vent—in the absence of a tube the atmospheric vent is the representative left-oral hose connection 314, and when a tube is coupled the atmospheric vent is the left oral prong 334.

In addition to or in place of the conserver 306 monitoring rise time and/or peak pressure, the conserver 306 may also determine the presence or absence of a tube connected to the conserver 306 based on fall time of the pressure. Still considering the representative left-oral hose connection 314 and the left oral tube 336, again the conserver 306 may test by providing the burst of therapeutic gas to the left-oral hose connection 314, but in this example the presence or absence of the tube may be determined based on pressure decay or fall time of the pressure measured. In the absence of a tube coupled to the representative left-oral hose connection 314, after the burst of the therapeutic gas ends the pressure falls at a first rate owing to the atmospheric vent being the left-oral hose connection 310. However, when a tube is coupled to the representative left-oral hose connection 314, after the burst of therapeutic gas ends the pressure falls at a second rate, slower than the first rate, with the slower second rate attributable to the resistance to gas flow presented by the tube in combination with the “capacitive” effects of the tubing.

Thus, by testing for the presence of a tube connected to each hose connection, the example conserver 306 may be informed as to the type of operation to implement. In the presence of nasal-only sensing, the conserver 306 may be designed and constructed to act only with respect to the nares. In the presence of both nasal sensing and oral sensing, the conserver 306 may be designed and constructed to implement ambulatory operation during wakefulness, and implement either the CPAP non-compliance therapy discussed above or the wakefulness to sleep state detection and increased volume delivery discussed above. In the presence oral-only sensing, the conserver 306 may be designed and constructed to implement the wakefulness to sleep state detection and increased volume delivery discussed above. The specification now turns to an example implementation of the conserver 306.

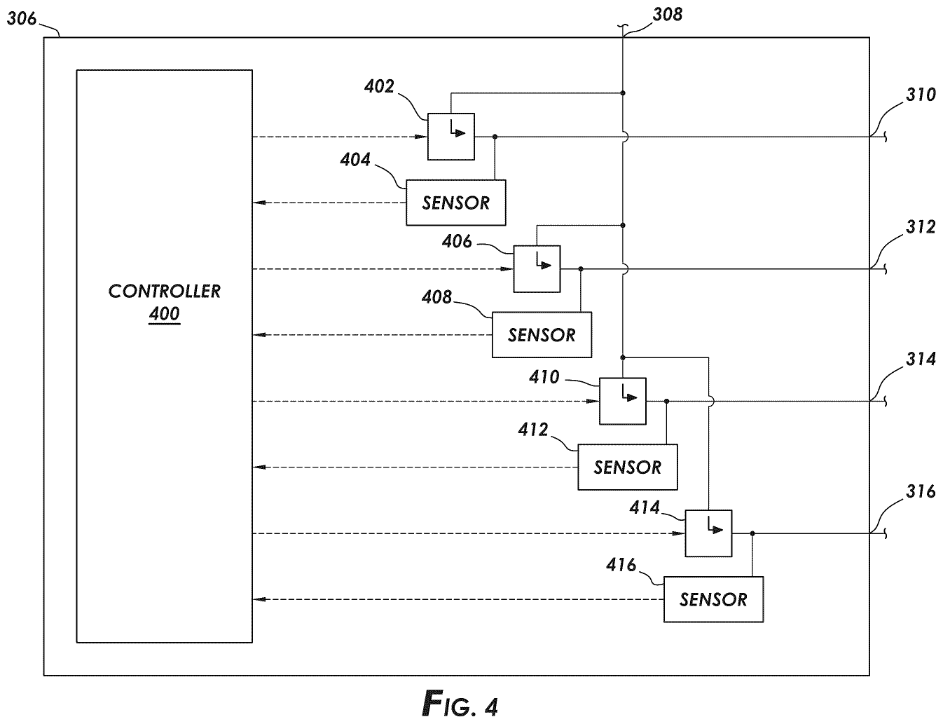

FIG. 4 shows an example conserver 306. The example conserver 306 defines the source-house connection 308, the right-naris hose connection 310, the left-naris hose connection 312, the left-oral hose connection 314, and the right-oral hose connection 316. Internally, the example conserver 306 defines both electrical connections and the fluidic connections (e.g., with therapeutic gas considered a fluid). In order to differentiate between electrical connections and fluidic connections, FIG. 4 illustrates electrical connections between components with dashed lines, and fluidic connections (e.g., tubing connections between devices) with solid lines.

The example conserver 306 of FIG. 4 comprises a controller 400. The example controller 400 may drive on/off or Boolean signals, such as signals to control the state of various electrically-controlled valves. In addition to or in place of the Boolean signals, the controller 400 may drive analog signals, such as analog signals to control proportional valves that define or control flow rate of gas through the proportional valve based on the amplitude of the analog signal. Moreover, the example controller 122 may read signals from various sensors (e.g., analog signals, digital signals) indicative of inhalations and exhalations through the breathing orifices of the patient, and signals indicative of therapeutic gas flow supplied to the hose connections. The controller 400 may take any suitable form. In some cases, the controller 400 may be constructed of individual circuit components (e.g. an analog implementation). In other cases, the controller 400 may be an application specific integrated circuit (ASIC), a microcontroller, a processor, a digital signal processor (DSP), a programmable logic device (PLD), a field programmable gate array (FPGA), or a programmable system-on-a-chip (PSOC). In yet still other cases, the controller 400 may be a combination of individual circuit components and/or an ASIC, and one of the programmable elements (processor, microcontroller, DSP, PLD, FPGA, and PSOC). In any event, the controller 400 is designed and constructed to read inputs and drive outputs responsive to the inputs to implement the various features and therapies discussed herein.

The example controller 306 includes an electrically-controlled valve 402. Electrically-controlled valve 402 is electrically coupled to the controller 400. The electrically-controlled valve 402 defines a first port coupled to the source-hose connection 308 and a second port coupled to the right-naris hose connection 310. The electrically-controlled valve 402 is configured to fluidly couple the source-hose connection 308 to the right-naris hose connection 310 responsive to an electrical signal from the controller 400. In one example, the electrically-controlled valve 402 is a five-volt solenoid operated valve that selectively couples an inlet port to an outlet or common port, such as a part number R434007035, 5 Volt, 1.3 Watt, solenoid operated valve available from Aventics of Laatzen, Germany. In another example, the electrically-controlled valve 402 is a proportional valve that not only controls the flow the therapeutic gas in an on-off sense, but also controls the flow rate through the valve based on a control signal provided from the controller 400 (e.g., to control the flow rate in the continuous-flow mode or the pulse-flow continuous mode). An example of a suitable proportional valve is a part number PV10PM1208025 from Humphrey Products of Kalamazoo, MI.

The example controller 306 further includes a right-narial sensor 404 fluidly coupled to the right-narial hose connection 310, and electrically coupled to the controller 400. The right-narial sensor 404 is designed and constructed to sense an attribute of airflow associated with the right-narial hose connection 310 (and thus the right naris), and to provide to the controller 400 signal indicative of right-narial airflow. In one example, the right-narial sensor 404 is a pressure sensor designed and constructed to produce a signal responsive to the range of sensed inhalation pressured (e.g., pressure below zero PSIG) and sensed exhalation pressure (e.g., pressure above zero PSIG). An example full range pressure may be a part number ESCP-BMS1+/−1 Bar from Servoflo Corporation of Lexington, MA. Thus, based on receiving a signal indicative of the right-narial airflow, the controller 400 may discern transitions from wakefulness to sleep, and/or determine the presence or absence of sleep apnea events.

In other cases, the right-narial sensor 404 may be a pressure sensor designed and constructed to be particularly sensitive to the transition between the end of an exhalation and the beginning of an inhalation, such as the sensor and operation described in: commonly assigned U.S. Pub. 2022/0176059 titled “System, Method and Apparatus for Dynamic Oxygen Conserver with Inhalation Sensor” having the same inventor as the current specification; and commonly assigned U.S. application Ser. No. 17/854,886 filed Jun. 30, 2022 titled “Methods and Systems of Supplying Therapeutic Gas” having the same inventor as the current specification. Both of these applications are incorporated by reference herein as if reproduced in full below. In such a case, while being particularly sensitive to the pressure at the transition between the end of exhalation and the beginning of inhalation, the sensor may be less sensitive or not sensitive to the peak inhalation and peak exhalation airflows. Nevertheless, in being able to determine transitions between the end of exhalation and the beginning of inhalation, the controller 400 may discern transitions from wakefulness to sleep, and/or determine the presence or absence of sleep apnea events.

In yet still further cases, the right-narial sensor 404 may be a combination of a sensor designed and constructed to sense the range of inhalation and exhalation pressure, as well as a sensor designed and constructed to be more sensitive to the transition between exhalation and inhalation. In such cases, discerning transitions from wakefulness to sleep and/or determining the presence or absence of sleep apnea events may be based on the readings of the extended-range sensor, and inhalation detection for purposes of delivering therapeutic gas in the bolus-flow mode and/or the pulse-flow continuous mode may be by way of the transition-sensitive sensor. Regardless of the precise arrangement, the controller 400 may read a signal indicative right-narial airflow from the right narial sensor 404, and selectively deliver therapeutic gas to the right naris by commanding the electrically-controlled valve 402 to deliver the therapeutic gas. The delivery may be in bolus-flow mode, continuous-flow mode, pulse-flow continuous mode, or by pulsing the flow the therapeutic gas (e.g., at a pulse rate of three pulses per second or more).

During periods of time when the controller 306 provides a burst of therapeutic gas for purposes of detecting the presence or absence of a tube connected to the right-narial hose connection 310, the right-narial sensor 404 may also provide the signal indicative of gas flow. In one example, the signal indicative of gas flow may be provided from the same output port of the right-narial sensor 404 that produces the signal indicative of right-narial airflow (e.g., same signal, just different periods of time). A right-narial sensor 404 sensitive to the range of the right-narial airflow may also have a range wide enough to capture the range of applied pressure of the therapeutic gas. However, even if the range of the right-narial sensor 404 is less than the range of the applied pressure, the controller 400 may nevertheless be able to determine rise times and/or fall times associated with the burst of therapeutic gas as part of making the determination as to the presence or absence of the tube connected to the right-naris hose connection 310. Even the right-narial sensor 404 in the form particularly sensitive to the transition between exhalation and inhalation may be used by the controller 400 by monitoring the portion of the rise time present in the signal indicative of gas flow, the portion of the fall time present in the signal indicative of the fall time, and/or the time duration between zero readings (e.g., the time duration between zero readings will be shorter in the absence of a tube, and longer in the presence of a tube). Thus, any or all the variations associated with the right-narial sensor 404 may also be used in detecting the presence or absence of the tube connected to right-naris hose connection 310.

Still referring to FIG. 4, the example controller 306 further includes an electrically-controlled valve 406. Electrically-controlled valve 406 is electrically coupled to the controller 400. The electrically-controlled valve 406 defines a first port coupled to the source-hose connection 308 and a second port coupled to the left-naris hose connection 312. The electrically-controlled valve 406 is configured to fluidly couple the source-hose connection 308 to the left-naris hose connection 312 responsive to an electrical signal from the controller 400. The example electrically-controlled valve 406 may be a solenoid operated valve as described above or a proportional valve as described above.

The example controller 306 further includes a left-narial sensor 408 fluidly coupled to the left-narial hose connection 312, and electrically coupled to the controller 400. The left-narial sensor 408 is designed and constructed to sense an attribute of airflow associated with the left-narial hose connection 312 (and thus the left naris), and to provide to the controller 400 a signal indicative of left-narial airflow. In one example, the left-narial sensor 408 is a pressure sensor designed and constructed to produce a signal responsive to the range of sensed inhalation pressured and sensed exhalation pressure. In other cases, the left-narial sensor 408 may be a pressure sensor designed and constructed to be particularly sensitive to the transition between the end of exhalation and the beginning of inhalation, such as described in the commonly assigned applications noted above having the same inventor as the current specification. In yet still further cases, the left-narial sensor 408 may be a combination of an extended-range sensor and a transition-sensitive sensor. Regardless, by reading the signal(s) indicative left-narial airflow, the controller 400 may determine the presence or absence of a tube connected to the left-naris hose connection 312, discern transitions from wakefulness to sleep, and/or determine the presence or absence of sleep apnea events. It further follows then that the controller 400 may read a signal indicative left-narial airflow from the left-narial sensor 408, and selectively deliver therapeutic gas to the left naris by commanding electrically-controlled valve 406 to deliver the therapeutic gas. The delivery may be in bolus-flow mode, continuous-flow mode, pulse-flow continuous mode, or by pulsing the flow the therapeutic gas (e.g., at a pulse rate of three pulses per second or more).

The example controller 306 further includes an electrically-controlled valve 410. Electrically-controlled valve 410 is electrically coupled to the controller 400. The electrically-controlled valve 410 defines a first port coupled to the source-hose connection 308 and a second port coupled to the left-oral hose connection 314. The electrically-controlled valve 410 is configured to fluidly couple the source-hose connection 308 to the left-oral hose connection 314 responsive to an electrical signal from the controller 400. The example electrically-controlled valve 410 may be a solenoid operated valve as described above or a proportional valve as described above.

The example controller 306 further includes a left-oral sensor 412 fluidly coupled to the left-oral hose connection 314, and electrically coupled to the controller 400. The left-oral sensor 412 is designed and constructed to sense an attribute of airflow associated with the left-oral hose connection 312 (and thus the left mouth), and to provide to the controller 400 a signal indicative of left-oral airflow. In one example, the left-oral sensor 412 is a pressure sensor designed and constructed to produce a signal responsive to the range of sensed inhalation pressured and sensed exhalation pressure. In other cases, the left-oral sensor 412 may be a pressure sensor designed and constructed to be particularly sensitive to the transition between the end of exhalation and the beginning of inhalation, such as described in the commonly assigned applications noted above having the same inventor as the current specification. In yet still further cases, the left-oral sensor 412 may be a combination of a full-range sensor and a transition-sensitive sensor. Regardless, by reading the signal(s) indicative left-oral airflow, the controller 400 may determine the presence or absence of a tube connected to the left-oral hose connection 314, discern transitions from wakefulness to sleep, and/or determine the presence or absence of sleep apnea events. It further follows then that the controller 400 may read a signal indicative left-oral airflow from the left-oral sensor 412, and selectively deliver therapeutic gas to the left mouth by commanding electrically-controlled valve 410 to deliver the therapeutic gas. The delivery may be in bolus-flow mode, continuous-flow mode, pulse-flow continuous mode, or by pulsing the flow the therapeutic gas (e.g., at a pulse rate of three pulses per second or more).

Still referring to FIG. 4, the example controller 306 further includes an electrically-controlled valve 414. Electrically-controlled valve 414 is electrically coupled to the controller 400. The electrically-controlled valve 414 defines a first port coupled to the source-hose connection 308 and a second port coupled to the right-oral hose connection 316. The electrically-controlled valve 414 is configured to fluidly couple the source-hose connection 308 to the right-oral hose connection 316 responsive to an electrical signal from the controller 400. The example electrically-controlled valve 414 may be a solenoid operated valve as described above or a proportional valve as described above.

The example controller 306 further includes a right-oral sensor 416 fluidly coupled to the right-oral hose connection 316, and electrically coupled to the controller 400. The right-oral sensor 416 is designed and constructed to sense an attribute of airflow associated with the right-oral hose connection 314 (and thus the right mouth), and to provide to the controller 400 a signal indicative of right-oral airflow. In one example, the right-oral sensor 416 is a pressure sensor designed and constructed to produce a signal responsive to the full range of sensed inhalation pressured and sensed exhalation pressure. In other cases, the right-oral sensor 416 may be a pressure sensor designed and constructed to be particularly sensitive to the transition between the end of exhalation and the beginning of inhalation, such as described in the commonly assigned applications noted above having the same inventor as the current specification. In yet still further cases, the right-oral sensor 416 may be a combination of a full-range sensor and a transition-sensitive sensor. Regardless, by reading the signal(s) indicative right-oral airflow, the controller 400 may determine the presence or absence of a tube connected to the right-oral hose connection 316, discern transitions from wakefulness to sleep, and/or determine the presence or absence of sleep apnea events. It further follows then that the controller 400 may read a signal indicative right-oral airflow from the right-oral sensor 416, and selectively deliver therapeutic gas to the right mouth by commanding electrically-controlled valve 41r to deliver the therapeutic gas. The delivery may be in bolus-flow mode, continuous-flow mode, pulse-flow continuous mode, or by pulsing the flow the therapeutic gas (e.g., at a pulse rate of three pulses per second or more).

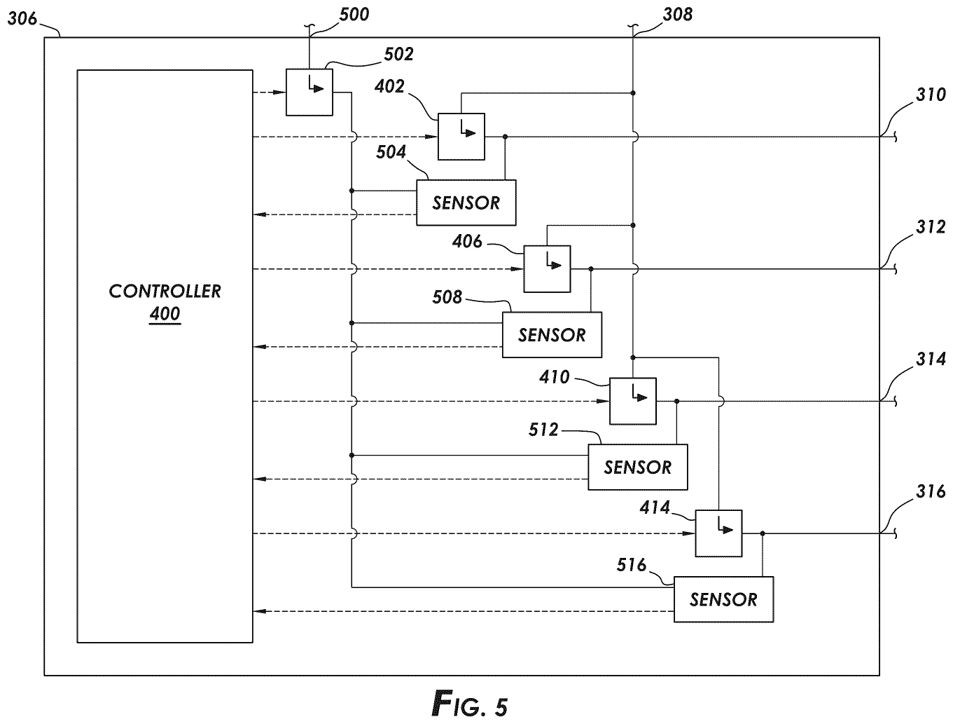

FIG. 5 shows another example conserver 306. The example conserver 306 of FIG. 5 defines the source-hose connection 308, the right-naris hose connection 310, the left-naris hose connection 312, the left-oral hose connection 314, and the right-oral hose connection 316, and additionally defines an atmospheric vent 500. As before, FIG. 5 illustrates electrical connections between components with dashed lines, and fluidic connections (e.g., tubing connections between devices) with solid lines. Components in the example conserver 306 of FIG. 5 that are duplicative of the conserver of FIG. 4 carry the same reference number, and will not be described again in detail so as not to unduly lengthen the specification.

The example conserver 306 of FIG. 5 comprises the controller 400, as well as the electrically-controlled valves 402, 406, 410, and 414. The various valves may be fluidly and electrically connected as discussed with respect to FIG. 4, and may be on-off valves or proportional valves as discussed above. The example controller 306 of FIG. 5 further includes a right-narial sensor 504 fluidly coupled to the right-narial hose connection 310, and electrically coupled to the controller 400. The right-narial sensor 504 is designed and constructed to sense an attribute of airflow associated with the right-narial hose connection 310 (and thus the right naris), and to provide to the controller 400 signal indicative of right-narial airflow. In the example of FIG. 5, the right-narial sensor 404 is a flow sensor designed and constructed to produce a signal responsive to sensed airflow (e.g., inhalation and exhalation), such as part number DF6-P0010AM2 from Omron Corporation, Japan. Thus, based on receiving a signal indicative of the right-narial airflow, the controller 400 may discern transitions from wakefulness to sleep, and/or determine the presence or absence of sleep apnea events.

To sense airflow, the sensed airflow moves through right-narial sensor 504. However, during periods of time when the electrically-controlled valve 402 provides therapeutic gas to the right-naris hose connection 310, the therapeutic gas will also tend to flow through the right-narial sensor 504, bypassing the patient—hereafter the bypass flow. In order to block the bypass flow, the example controller 306 includes an electrically-controlled valve 502. The example electrically-controlled valve 502 has a first port that defines the atmospheric vent 500 and a second port fluidly coupled to the right-narial sensor 504. When the control signal to the electrically-controlled valve 502 is asserted, the electrically-controlled valve blocks 502 flow through the right-narial sensor 504 (and, as discussed more below, the other flow sensors). In this example then, electrically-controlled valve 502 is a normally-open valve. The controller 400 of this example is designed and constructed to block the flow through the right-narial sensor 504 during periods of therapeutic gas delivery (e.g., when the signal to the electrically-controlled valve 402 is asserted), as well as for a predetermined amount of time thereafter to enable the therapeutic gas stored in the “capacitive” aspects of the tubing of the cannula to reach the patient.

However, in the case of the controller 400 making a determination as to the presence or absence of a tube coupled to the right-naris hose connection 310, the controller 400 may operate the electrically-controlled valve 502 differently, or not all. In one example, when testing to determine the presence or absence of the tube coupled to the right-naris hose connection 310, the controller 400 may assert the control signal to the electrically-controlled valve 502 (i.e., block flow through the right-narial sensor 504) simultaneously with the assertion of the control signal to the electrically-controlled valve 402 (i.e., to enable flow through the valve). Thus, the burst of therapeutic gas is forced to flow out the right-naris hose connection 310 during delivery, but any therapeutic stored in the “capacitive” aspects of any attached tubing may backflow through right-narial sensor 504. It follows that the controller 400 may determine the presence or absence of the tube connected to the right-naris hose connection 310 based the amount of flow sensed after the delivery is ceased. Higher flow sensed is indicative of a tube connected to the right-naris hose connection 310, and lower flow sensed is indicative of the absence of a tube connected to the right-naris hose connection 310.

In another case, when testing to determine the presence or absence of the tube coupled to the right-naris hose connection 310, the controller 400 may refrain asserting the control signal to the electrically-controlled valve 502 during periods that the control signal to the electrically-controlled valve 402 is asserted. It follows that at least a portion of the burst of therapeutic gas flows through the right-narial sensor 504 during the burst. By measuring the flow of therapeutic gas during the burst, the controller 400 may determine the presence or absence of the tubing. For example, higher flow sensed is indicative of a tube connected to the right-naris hose connection 310 because of the higher resistance to airflow presented by the tube and/or the increased distance to the atmospheric vent of the right nasal prong 328 (FIG. 3). Oppositely, lower flow sensed is indicative of the absence of a tube because, in the absence of a tube, the right-naris hose connection 310 is an atmospheric vent. Thus, regardless of the use of a flow sensor rather than a pressure sensor, the controller 400 may nevertheless determine the presence or absence of tube connected to the right-naris hose connection 310.

In the example of FIG. 5, the right-narial sensor 504 is fluidly coupled between the electrically-controlled valve 402 and the right-naris hose connection 310. However, in other cases the electrically-controlled valve 402 may be a three-port valve. The source-hose connection 308 may couple to a first port, the right-narial sensor 504 may couple to a second port, and a common port may couple to the right-naris hose connection 310. Thus, the valve may selectively couple either the source-hose connection 308 to the right-naris hose connection 310 or the right-narial sensor 504 to the right-naris hose connection 310. During periods of time when the source-hose connection 310 is coupled to the right-naris hose connection 310, flow through the right-narial sensor 504 is blocked. However, in such cases the electrically-controlled valve 502 may still be implemented to further block the bypass flow associated with the “capacitive” aspects of the tubing of the nasal cannula.

Still referring to FIG. 5, the example controller 306 further includes a left-narial sensor 508 fluidly coupled to the left-narial hose connection 312, and electrically coupled to the controller 400. The left-narial sensor 508 is designed and constructed to sense an attribute of airflow associated with the left-narial hose connection 312 (and thus the left naris), and to provide to the controller 400 a signal indicative of left-narial airflow. In the example of FIG. 5, the left-narial sensor 508 is a flow sensor designed and constructed to produce a signal responsive to sensed airflow (e.g., inhalation and exhalation). The example left-narial sensor 508 is fluidly coupled to the electrically-controlled valve 502 to selectively block the flow through the left-narial sensor 508 as discussed above with respect to the right-narial sensor 504. Thus, based on receiving a signal indicative of the left-narial airflow, the controller 400 may determine the presence or absence of a tube coupled to the left-naris hose connection 312, discern transitions from wakefulness to sleep, and/or determine the presence or absence of sleep apnea events.

The example controller 306 further includes a left-oral sensor 512 fluidly coupled to the left-oral hose connection 314, and electrically coupled to the controller 400. The left-oral sensor 512 is designed and constructed to sense an attribute of airflow associated with the left-oral hose connection 314 (and thus the left mouth), and to provide to the controller 400 a signal indicative of left-mouth airflow. In the example of FIG. 5, the left-oral sensor 512 is a flow sensor designed and constructed to produce a signal responsive to sensed airflow (e.g., inhalation and exhalation). The example left-oral sensor 512 is fluidly coupled to the electrically-controlled valve 502 to selectively block the flow through the left-oral sensor 512 as discussed above with respect to the right-narial sensor 504. Thus, based on receiving a signal indicative of the left-oral airflow, the controller 400 may determine the presence or absence of a tube coupled to the left-oral hose connection 314, discern transitions from wakefulness to sleep, and/or determine the presence or absence of sleep apnea events.

Still referring to FIG. 5, the example controller 306 further includes a right-oral sensor 516 fluidly coupled to the right-oral hose connection 316, and electrically coupled to the controller 400. The right-oral sensor 516 is designed and constructed to sense an attribute of airflow associated with the right-oral hose connection 316 (and thus the right mouth), and to provide to the controller 400 a signal indicative of right-mouth airflow. In the example of FIG. 5, the right-oral sensor 516 is a flow sensor designed and constructed to produce a signal responsive to sensed airflow (e.g., inhalation and exhalation). The example right-oral sensor 516 is fluidly coupled to the electrically-controlled valve 502 to selectively block the flow through the right-oral sensor 516 as discussed above with respect to the right-narial sensor 504. Thus, based on receiving a signal indicative of the right-oral airflow, the controller 400 may determine the presence or absence of a tube coupled to the left-oral hose connection 314, discern transitions from wakefulness to sleep, and/or determine the presence or absence of sleep apnea events.

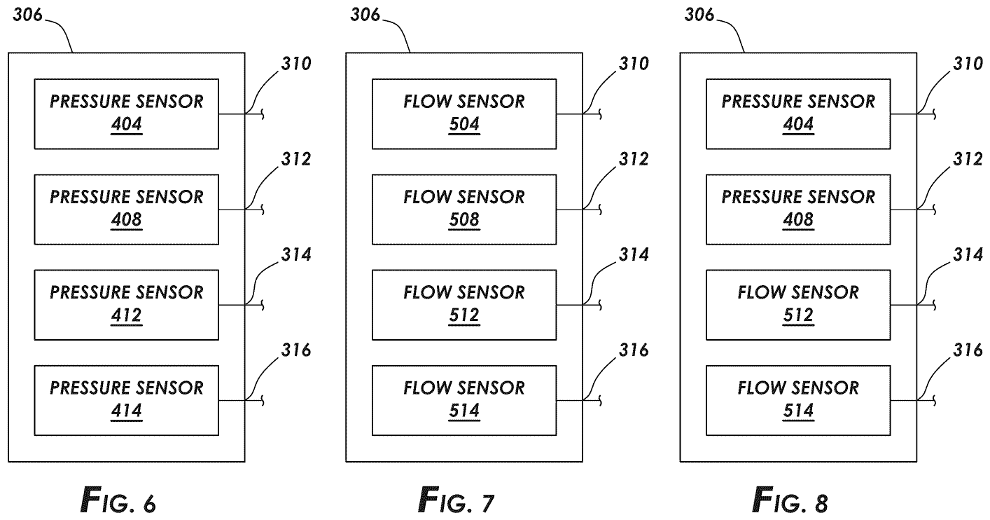

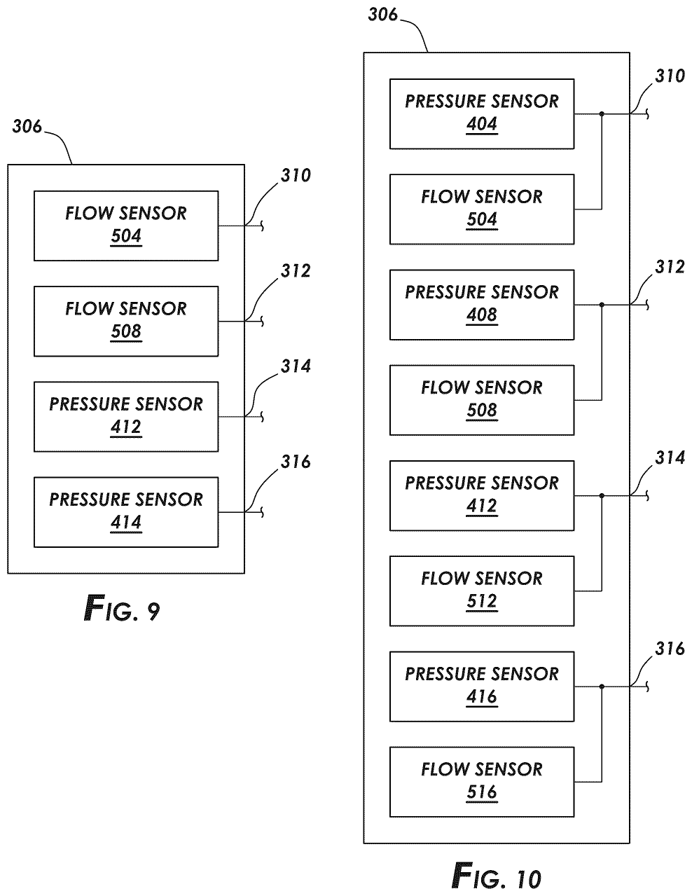

FIG. 6 shows, in block diagram form, the conserver 306 of FIG. 4 in a shorthand notation. In particular, FIG. 6 shows the conserver 306 comprising the right-narial sensor 404 in the form of a pressure sensor, the left-narial sensor 408 in the form of a pressure sensor, the left-oral sensor 412 in the form of a pressure sensor, and the right-oral sensor 414 in the form of a pressure sensor. FIG. 7 shows, in block diagram form, the conserver 306 of FIG. 5 in a shorthand notation. In particular, FIG. 7 shows the conserver 306 comprising the right-narial sensor 504 in the form of a flow sensor, the left-narial sensor 508 in the form of a flow sensor, the left-oral sensor 512 in the form of a flow sensor, and the right-oral sensor 514 in the form of a flow sensor. The shorthand notations of FIGS. 6 and 7 are thus used to present variants of the conserver 306. For example, FIG. 8 shows an example conserver 306 in which the narial sensors are pressure sensors, and the oral sensors are flow sensors. FIG. 9 shows an example conserver 306 in which the narial sensors are flow sensors, and the oral sensors are pressure sensors. FIG. 10 shows an example conserver 306 having both pressure and flow sensors associated with each hose connection.

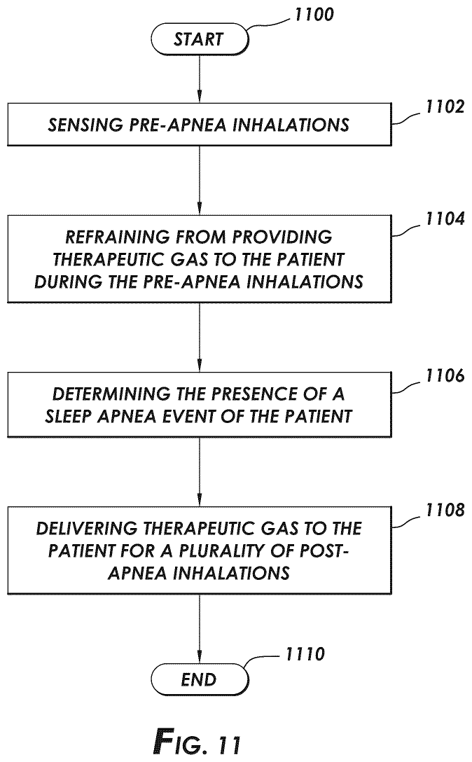

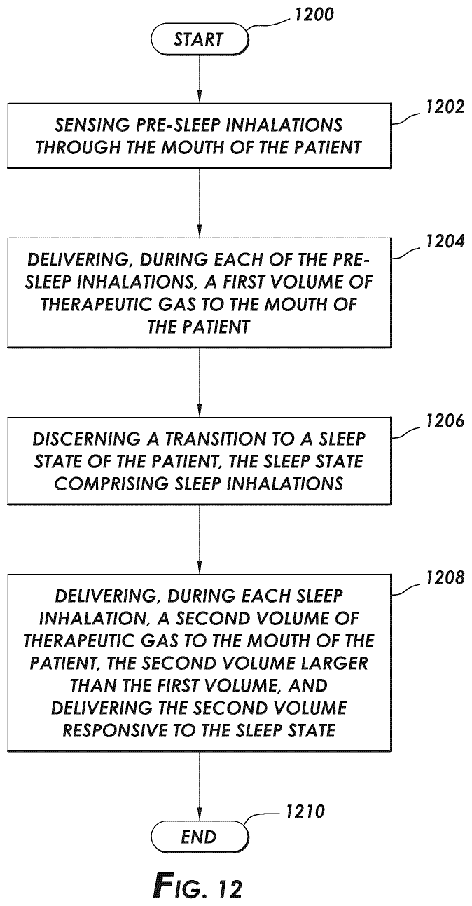

FIG. 11 shows a method accordance with at least some embodiments. The example method may be implemented by the controller 400 (FIG. 4), such as by instructions on one of the programmable elements (processor, microcontroller, DSP, PLD, FPGA, PSOC). In particular, the method starts (block 1100) and comprises: sensing pre-apnea inhalations (block 1102); refraining from providing therapeutic gas to the patient during the pre-apnea inhalations (block 1104); determining the presence of a sleep apnea event of the patient (block 1106); and then delivering therapeutic gas to the patient for a plurality of post-apnea inhalations (block 1108). Thereafter the method may end (block 1110), likely to be restarted on the next inhalation or set of inhalations.

FIG. 12 shows a method accordance with at least some embodiments. The example method may be implemented by the controller 400 (FIG. 4), such as by instructions on one of the programmable elements (processor, microcontroller, DSP, PLD, FPGA, PSOC). In particular, the method starts (block 1200) and comprises: sensing pre-sleep inhalations through the mouth of the patient (block 1202); delivering, during each of the pre-sleep inhalations, a first volume of therapeutic gas to the mouth of the patient (block 1204); discerning a transition to a sleep state of the patient, the sleep state comprising sleep inhalations (block 1206); and responsive to the sleep state delivering, during each sleep inhalation, a second volume of therapeutic gas to the mouth of the patient, the second volume larger than the first volume (block 1208). Thereafter the method may end (block 1210), likely to be restarted on the next inhalation or set of inhalations.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A method of providing therapeutic gas to a patient, the method comprising:
sensing, by a delivery device, pre-apnea inhalations;
refraining, by the delivery device, from providing therapeutic gas to the patient during the pre-apnea inhalations;
determining, by the delivery device, a presence of a sleep apnea event of the patient; and then
delivering, by the delivery device, therapeutic gas to the patient for a plurality of post-apnea inhalations.

2. The method of claim 1 wherein sensing the pre-apnea inhalations further comprises sensing by way of a cannula comprising oral sensing tubes that are bifurcated.

3. The method of claim 2 wherein sensing by way of the cannula comprising oral sensing tubes further comprises sensing by way of the cannula comprising narial sensing tubes that are bifurcated.

4. The method of claim 1 further comprising, after determining the presence of the sleep apnea event and before the post-apnea inhalations, delivering therapeutic gas to at least one of the nares and the mouth.

5. The method of claim 1 further comprising, during the sleep apnea event, delivering therapeutic gas in a plurality of pulses to at least one the nares and the mouth.

6. The method of claim 5 wherein delivering therapeutic gas in the plurality of pulses further comprises pulsing at a pulse rate of a two pulses per second or greater.

7. The method of claim 1 delivering therapeutic gas for the plurality of post-apnea inhalations further comprises delivering therapeutic in a continuous-flow mode for a predetermined period of time.

8. The method of claim 1 delivering therapeutic gas for the plurality of post-apnea inhalations further comprises delivering therapeutic gas as a bolus during each of the plurality of post-apnea inhalations.

9. The method of claim 1 wherein, after the plurality of post-apnea inhalations, the method further comprises:
ceasing delivery of therapeutic gas; and then
refraining from delivering therapeutic gas until a subsequent sleep apnea event;
determining, by the delivery device, a presence of the subsequent sleep apnea event of the patient; and then
delivering, by the delivery device, therapeutic gas to the patient for a plurality of second post-apnea inhalations.

10. A therapeutic gas delivery device comprising:
a controller;
a source-hose connection, a first nose connection, a first mouth connection, and a second mouth connection;
a first narial sensor electrically coupled to the controller and fluidly coupled to the first nose connection, the first narial sensor configured to create a first signal indicative of narial airflow;
a first valve electrically coupled to the controller and configured to fluidly couple the source-hose connection to the first nose connection;
a first mouth sensor electrically coupled to the controller and fluidly coupled to the first mouth connection, the first mouth sensor configured to create a first signal indicative of oral airflow;
a second valve electrically coupled to the controller and configured to fluidly couple the source-hose connection to the first mouth connection;
a second mouth sensor electrically coupled to the controller and fluidly coupled to the second mouth connection, the second mouth sensor configured to create a second signal indicative of oral airflow;
a third valve electrically coupled to the controller and configured to fluidly couple the source-hose connection to the second mouth connection;
the controller is configured to:
read at least one of the first signal indicative of narial airflow, the first signal indicative of oral airflow, and second signal indicative of oral airflow;
determine, based on the reading, a presence or absence of a sleep apnea;
refrain, in the absence of sleep apnea, from delivering therapeutic gas; and
command, during a plurality of subsequent inhalations after the presence of sleep apnea is detected, at least one of the first, second, and third valves to deliver therapeutic gas to their respective connections.

11. The therapeutic gas delivery device of claim 10 further comprising:
a second nose connection;
a second narial sensor electrically coupled to the controller and fluidly coupled to the second nose connection, the second narial sensor configured to create a second signal indicative of narial airflow;
a fourth valve electrically coupled to the controller and configured to fluidly couple the source-hose connection to the second nose connection;
wherein when the controller reads, the controller is further configured to read at least the second signal indicative of narial airflow;
wherein when the controller commands, the controller is further configured to command at least the fourth valve to deliver therapeutic gas.

12. The therapeutic gas delivery device of claim 10 wherein the controller is further configured to, after a determination of the presence of sleep apnea and before an immediately subsequent inhalation, command at least one of the first, second, and third valves to deliver therapeutic gas to their respective connections.

13. The therapeutic gas delivery device of claim 12 wherein when the controller commands delivery of therapeutic gas during the presence of sleep apnea, the controller is further configured to deliver for at least one selected from a group consisting of: a length of a previous sleep apnea event; three seconds; and greater than half a duration of a previous inhalation.

14. The therapeutic gas delivery device of claim 10 wherein the controller is further configured to, after a determination of the presence of sleep apnea and before the plurality of subsequent inhalations, pulse at least one of the first, second, and third valves to deliver pulses of therapeutic gas to their respective connections.

15. The therapeutic gas delivery device of claim 10 wherein the controller is further configured to, during the plurality of subsequent inhalations, command at least one of the first, second, and third valves to deliver therapeutic gas to their respective connections in a continuous-flow mode.

16. The therapeutic gas delivery device of claim 10 wherein the controller is further configured to, during the plurality of subsequent inhalations, command at least one of the first, second, and third valves to deliver therapeutic gas to their respective connections in a bolus-flow mode.

17. The therapeutic gas delivery device of claim 10 wherein the controller is further configured to, after the plurality of subsequent inhalations:
cease delivery of therapeutic gas; and
refrain from delivery of therapeutic gas until a subsequent sleep apnea event; and then
during a plurality of second subsequent inhalations after the presence of sleep apnea is detected, command at least one of the first, second, and third valves to deliver therapeutic gas to their respective connections.

18. A therapeutic gas delivery system comprising:
a source of therapeutic gas;
a cannula comprising:
a left narial prong fluidly coupled to a distal end of a left narial tube;
a right narial prong fluidly coupled to a distal end of a right narial tube, the right narial tube fluidly isolated from the left narial tube;
the left and right narial prongs define a first spacing;
a left oral prong fluidly coupled to a distal end of a left oral tube;
a right oral prong fluidly coupled to a distal end of a right oral tube, the right oral tube fluidly isolated from the left oral tube; and
the left and right oral prongs define a second spacing greater than the first spacing;
a therapeutic gas delivery device comprising a source-hose connection coupled to the source of therapeutic gas, a first nose connection coupled to the left narial tube, a second nose connection coupled to the right narial tube, a first mouth connection coupled to the left oral tube, and a second mouth connection coupled to the right oral tube, the therapeutic gas delivery device configured to:
create a signal indicative of left-narial airflow, a signal indicative of right-narial airflow, a signal indicative of left-oral airflow, and a signal indicative of right-oral airflow;
determine a presence or absence of sleep apnea based at least one of the signal indicative of left-narial airflow, the signal indicative of right-narial airflow, the signal indicative of left-oral airflow, and the signal indicative of right-oral airflow;
refrain, in the absence of sleep apnea, from delivering therapeutic gas to the cannula; and
deliver, during a plurality of post-apnea inhalations after the presence of sleep apnea is detected, therapeutic gas to at least one of the left narial prong, the right narial prong, the left oral prong, and the right oral prong.

19. The therapeutic gas delivery device of claim 18 wherein the therapeutic gas delivery device is further configured to, after a determination of the presence of sleep apnea and before a post-apnea inhalation, deliver therapeutic gas to at least one of the left narial prong, the right narial prong, the left oral prong, and the right oral prong.

20. The therapeutic gas delivery device of claim 19 wherein when the therapeutic gas delivery device delivers therapeutic gas during the presence of sleep apnea, the therapeutic gas delivery device is further configured to deliver for at least one selected from a group consisting of: a duration equal to a duration of a previous sleep apnea event; three seconds; and greater than half a duration of a previous inhalation.

21. The therapeutic gas delivery device of claim 18 wherein the therapeutic gas delivery device is further configured to, after a determination of the presence of sleep apnea and before a post-apnea inhalation, provide pulses of therapeutic gas to at least one of the left narial prong, the right narial prong, the left oral prong, and the right oral prong.

22. The therapeutic gas delivery device of claim 18 wherein the therapeutic gas delivery device is further configured to, during the plurality of post-apnea inhalations, deliver therapeutic gas in a continuous-flow mode to at least one of the left narial prong, the right narial prong, the left oral prong, and the right oral prong.

23. The therapeutic gas delivery device of claim 18 wherein the therapeutic gas delivery device is further configured to, during the plurality of post-apnea inhalations, deliver therapeutic gas in a bolus-flow mode to at least one of the left narial prong, the right narial prong, the left oral prong, and the right oral prong.

24. The therapeutic gas delivery device of claim 18 wherein the therapeutic gas delivery device is further configured to, after the plurality of post-apnea inhalations:
cease delivery of therapeutic gas;
refrain from delivery of therapeutic gas until a subsequent sleep apnea event; and then
during a plurality of second post-apnea inhalations, deliver therapeutic gas to at least one of the left narial prong, the right narial prong, the left oral prong, and the right oral prong.

25.-64. (canceled)
